(19) United States
(12) Patent Application Publication
(10) Pub. No.: US 2023/0018499 A1
(43) Pub. Date: Jan. 19, 2023

(54) Deep Learning Based Approach For OCT Image Quality Assurance

(71) Applicant: LightLab Imaging, Inc., Westford, MA (US)

(72) Inventors: Justin Akira Blaber, Lowell, MA (US); Ajay Gopinath, Bedford, MA (US); Humphrey Chen, Acton, MA (US); Kyle Edward Savidge, Medford, MA (US); Angela Zhang, Stow, MA (US); Gregory Patrick Amis, Westford, MA (US)

(73) Assignee: LightLab Imaging, Inc., Westford, MA (US)

(21) Appl. No.: 17/862,991

(22) Filed: Jul. 12, 2022

Related U.S. Application Data
(60) Provisional application No. 63/220,722, filed on Jul. 12, 2021.

(51) Int. Cl. G06T 7/00; G16H 50/20; G16H 30/40

(52) U.S. Cl. CPC G06T 7/0012; G16H 50/20; G16H 30/40; G06T 2207/10101; G06T 2207/30101; G06T 2207/30021

(57) ABSTRACT

Aspects of the disclosure relate to systems, methods, and algorithms to train a machine learning model or neural network to classify OCT images. The neural network or machine learning model can receive annotated OCT images indicating which portions of the OCT image are blocked and which are clear as well as a classification of the OCT image as clear or blocked. After training, the neural network can be used to classify one or more new OCT images. A user interface can be provided to output the results of the classification and summarize the analysis of the one or more OCT images.

### CROSS-REFERENCE TO RELATED APPLICATIONS

The present application claims the benefit of the filing date of U.S. Provisional Application No. 63/220,722, filed Jul. 12, 2021, the disclosure of which is hereby incorporated herein by reference.

### FIELD

The disclosure relates generally to the field of vascular system imaging and data collection systems and methods. In particular, the disclosure relates to methods of improving the detection of image quality and categorization of images in Optical Coherence Tomography (OCT) systems.

### BACKGROUND

Optical Coherence Tomography (OCT) is an imaging technique which uses light to capture cross-sectional images of tissue on the micron scale. OCT can be a catheter-based imaging modality that uses light to peer into coronary or other artery walls and generate images thereof for study. Utilizing coherent light, interferometry, and micro-optics, OCT can provide video-rate in-vivo tomography within a diseased vessel with micrometer level resolution. Viewing subsurface structures with high resolution using fiber-optic probes makes OCT especially useful for minimally invasive imaging of internal tissues and organs. This level of detail made possible with OCT allows a physician to diagnose as well as monitor the progression of coronary artery disease.

OCT images can be degraded for a variety of reasons. For example, an OCT image can be degraded due to the presence of blood within a vessel when an OCT image of that vessel is obtained. The presence of blood can block proper identification of vessel boundaries during intravascular procedures. Images which are degraded may not be useful for interpretation or diagnosis. For example, during a “pull-back,” a procedure in which an OCT device is used to scan the length of a vessel, thousands of images may be obtained, some of which may be degraded, inaccurate, or not useful for analysis due to the presence of blood blocking the lumen contour during the OCT pullback.

Identification of which OCT images are degraded requires a manual frame-by-frame or image-by-image analysis of hundreds or thousands of images obtained during an OCT scan of a vessel. Further, this analysis would be performed after the OCT procedure is complete, potentially requiring an additional OCT scan to obtain better quality images of portions of the vessel corresponding to the degraded images.

Additional equipment required to detect the presence of blood can change the typical clinical workflow, degrade image quality, or otherwise add complexity in clinical implementation. Other tools developed to detect potentially incorrect lumen detection have been shown to be unreliable and do not directly detect whether the OCT image captured was blood blocked and thus not useful for interpretation.

### SUMMARY

Real-time or near-real time identification of which images or group of images are degraded, directly from the images, would allow for those images to be ignored when interpreting the OCT scan and would allow for those portions of a vessel which were blocked to be rescanned while OCT equipment is still in situ.

Aspects of the disclosed technology allow for calculation of a clear image length (CIL) of an OCT pullback. A clear image length can be an indication on a contiguous section of an OCT pullback which is not obstructed, such as for example, by blood.

Aspects of the disclosed technology include a method of classifying a diagnostic medical image. The method can comprise receiving the diagnostic medical image; analyzing, in real time or near real time, with a trained machine learning model, the diagnostic medical image, wherein the trained machine learning model is trained on a set of annotated diagnostic medical images; identifying, based on the analyzing, an image quality for the diagnostic medical image; and outputting for display on a user interface, in real time or near real time, an indication of the identified image quality. The diagnostic medical image can a single image of a series of diagnostic medical images. The series of diagnostic medical images is obtained through an optical coherence tomography pullback. The diagnostic medical image can be classified as a first classification or a second classification. An alert or notification can be provided when the diagnostic medical image is classified in the second classification. The set of annotated diagnostic medical images cam annotations including clear, blood, or guide catheter. The diagnostic medical image can be a an optical coherence tomography image. The diagnostic medical image can be classified as a clear medical image or a blood medical image. A probability indicative of whether the diagnostic medical image is acceptable or not acceptable can be computed. A threshold method can be used to convert the computed probability to a classification of the diagnostic medical image. Graph cuts can be used to convert the computed probability to a classification of the diagnostic medical image. A morphological classification can be used to convert the computed probability to a classification of the diagnostic medical image. “Acceptable” can means that the diagnostic medical image is above a predefined threshold quality which allows for evaluation of characteristics of human tissue above a threshold level of accuracy or confidence. A clear image length or clear image length indicator can be displayed or outputted.

Aspects of the disclosed technology can include a system comprising a processing device coupled to a memory storing instructions, the instructions causing the processing device to: receive the diagnostic medical image; analyze, in real time or near real time, with a trained machine learning model, the diagnostic medical image, wherein the trained machine learning model is trained on a set of annotated diagnostic medical images; identify, based on the analyzing, an image quality for the diagnostic medical image; and output for display on a user interface, in real time or near real time, an indication of the identified image quality. The diagnostic medical image can be an optical coherence tomography (OCT) image. The instructions can be configured to display a plurality of OCT images along with an indicator associated with a classification of each image of the plurality of OCT images. The series of diagnostic medical images can be obtained through an optical coherence tomography pullback.

Aspects of the disclosed technology can include a non-transient computer readable medium containing program instructions, the instructions when executed perform the steps of receiving the diagnostic medical image; analyzing, in real time or near real time, with a trained machine learning model, the diagnostic medical image, wherein the trained machine learning model is trained on a set of annotated diagnostic medical images; identifying, based on the analyzing, an image quality for the diagnostic medical image; and outputting for display on a user interface, in real time or near real time, an indication of the identified image quality. The diagnostic medical image can be a single image of a series of diagnostic medical images. The series of diagnostic medical images can be obtained through an optical coherence tomography pullback. The diagnostic medical image can be classified as a first classification or a second classification. An alert or notification can be provided when the diagnostic medical image is classified in the second classification. The set of annotated diagnostic medical images cam annotations including clear, blood, or guide catheter. The diagnostic medical image can be classified as a clear medical image or a blood medical image. A probability indicative of whether the diagnostic medical image is acceptable or not acceptable can be computed. A threshold method can be used to convert the computed probability to a classification of the diagnostic medical image. Graph cuts can be used to convert the computed probability to a classification of the diagnostic medical image. A morphological classification can be used to convert the computed probability to a classification of the diagnostic medical image. “Acceptable” can mean that the diagnostic medical image is above a predefined threshold quality which allows for evaluation of characteristics of human tissue above a threshold level of accuracy or confidence. A clear image length or clear image length indicator can be displayed or outputted. An unclassifiable image can be stored to retrain the trained machine learning model.

### BRIEF DESCRIPTION OF THE DRAWINGS

FIG. 1 shows a schematic diagram of an imaging and data collection system in accordance with aspects of the disclosure.

FIG. 2A illustrates “clear” OCT images according to aspects of the disclosure.

FIG. 2B illustrates annotated “clear” OCT images according to aspects of the disclosure.

FIG. 3A illustrates “blocked” OCT images according to aspects of the disclosure.

FIG. 3B illustrates annotated “blocked” OCT images according to aspects of the disclosure.

FIG. 4 illustrates a histogram associated with a training set of data according to aspects of the disclosure.

FIG. 5 illustrates a flowchart of a training process according to aspects of the disclosure.

FIG. 6 illustrates a flowchart related to aspects of classifying OCT images according to aspects of the disclosure.

FIG. 7 illustrates aspects of techniques which can be used to classify or group a sequence of OCT images according to aspects of the disclosure.

FIG. 8 illustrates a user interface related to aspects of lumen contour confidence and image quality according to aspects of the disclosure.

FIG. 9 illustrates method which can be used to produce or calculate a clear image length (CIL) of an OCT pullback according to aspects of the disclosure.

FIG. 10 illustrates an example CIL cost matrix according to aspects of the disclosure.

FIG. 11 illustrates an example OCT pullback with a CIL incorporated into the OCT pullback according to aspects of the disclosure.

FIG. 12 is a flow diagram illustrating an example method of assuring image quality using a machine-learning task-based approach, according to aspects of the disclosure.

FIG. 13A is an example image from a lumen detection task, according to aspects of the disclosure.

FIG. 13B is an example task outcome for the image of FIG. 13A, according to aspects of the disclosure.

FIG. 13C is an example confidence result for the lumen detection task, according to aspects of the disclosure.

FIGS. 14A-B provide example graphs illustrating confidence values associated with FIG. 13C, according to aspects of the disclosure.

FIG. 15A is another example image from a lumen detection task, according to aspects of the disclosure.

FIG. 15B is another example task outcome for the image of FIG. 15A, according to aspects of the disclosure.

FIG. 15C is another example confidence result for the lumen detection task, according to aspects of the disclosure.

FIGS. 16A-B provide example graphs illustrating confidence values associated with FIG. 15C, according to aspects of the disclosure.

FIG. 17A illustrates an example confidence aggregation on an A-line-frame basis, according to aspects of the disclosure.

FIG. 17B illustrates an example confidence aggregation on a frame-pullback basis, according to aspects of the disclosure.

FIG. 18 is a screenshot of an example user interface according to aspects of the disclosure.

### DETAILED DESCRIPTION

The disclosure relates to systems, methods, and non-transitory computer readable medium to identify, in real time, medical diagnostic images of poor image quality through the use of machine learning based techniques. Non-limiting examples of medical diagnostic images include OCT images, intravascular ultrasound (IVUS) images, CT scans, or MRI scans. For example, an OCT image is received and analyzed with a trained machine learning model. In some examples, the trained machine learning model can output a probability after analyzing an image. In some examples, the output probability can be related to a probability of whether the image belongs to a particular category or classification. For example, the classification may relate to the quality of the obtained image, and/or whether the quality is sufficient to perform further processing or analysis. In some examples, the classification can be a binary classification, such as “acceptable/unacceptable,” or “clear/blocked.”

A machine learning model may be trained based on an annotated or marked set of data. The annoted or marked set of data can include classifications or identification of portions of an image. According to some examples, the set of training data may be marked or classified as “blood blocked” or “not blood blocked.” In some examples, the training data may marked as acceptable or unacceptable/blocked. In some examples, the set of data can include OCT images obtained during one or more OCT pullbacks. In some examples, one or more sets of training data can be chosen or stratified so that each set of training data has similar distributions of the classifications of data.

The training set of data can be manipulated, such as by augmenting, modifying, or changing the set of training data. Training of the machine learning model can also take place on the manipulated set of training data. In some examples, the use of augmented, modified, or changed training data can generalize the machine learning model and prevent overfitting of the machine learning model.

After categorization of an OCT image by a trained machine learning model or after obtaining a probability that an image belongs to a particular category, post-processing techniques can be used on the image before displaying information related to the image to a user. In some examples, the post-processing techniques can include rounding techniques, graph cuts, erosion, dilation, or other morphological methods. Additional information related to the analyzed OCT image can also be generated and used when displaying an output related to the OCT images to a user, such as for example, information indicating which OCT images were unacceptable or blocked.

As used in this disclosure, an OCT image or OCT frame can be used interchangeably. Further as used in this disclosure, and as would be understood by a person of skill in the art, an “unacceptable” or “blocked” OCT image is one in which the lumen and vascular wall is not clearly imaged due to the presence of blood or other fluid.

Although examples given here are primarily described in connection with OCT images, a person of skill in the art will appreciate that the techniques described herein can be applied to other imaging modulaties.

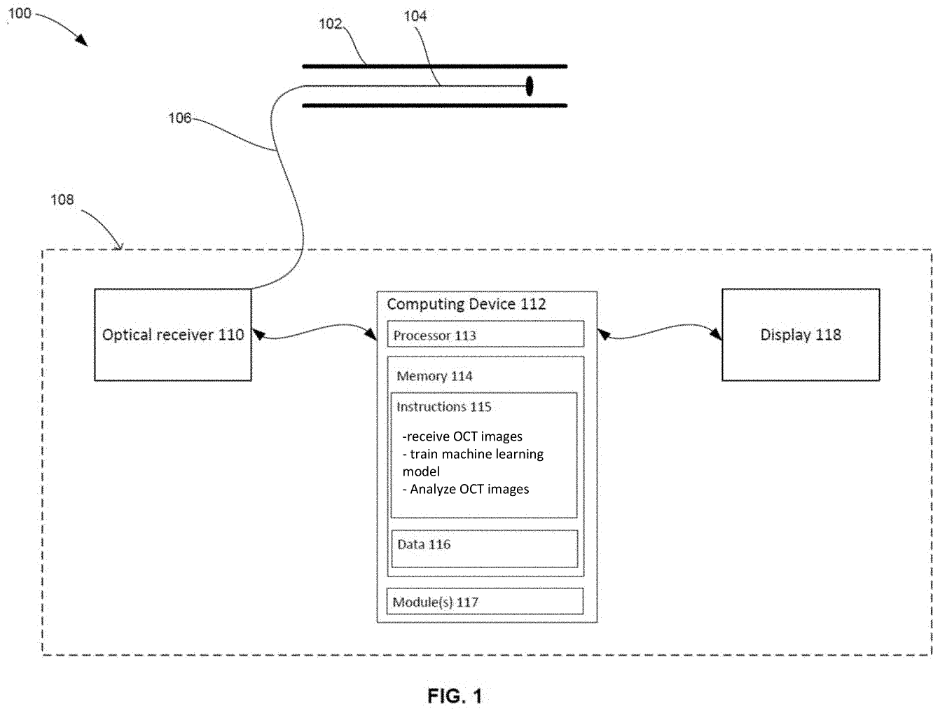

FIG. 1 illustrates a data collection system 100 for use in collecting intravascular data. The system may include a data collection probe 104 that can be used to image a blood vessel 102. A guidewire, not shown, may be used to introduce the probe 104 into the blood vessel 102. The probe 104 may be introduced and pulled back along a length of a blood vessel while collecting data. As the probe 104 is pulled back, or retracted, a plurality of scans or OCT and/or IVUS data sets may be collected. The data sets, or frames of image data, may be used to identify features, such as vessel dimensions and pressure and flow characteristics.

The probe 104 may be connected to a subsystem 108 via an optical fiber 106. The subsystem 108 may include a light source, such as a laser, an interferometer having a sample arm and a reference arm, various optical paths, a clock generator, photodiodes, and other OCT and/or IVUS components.

The probe 104 may be connected to an optical receiver 110. According to some examples, the optical receiver 110 may be a balanced photodiode based system. The optical receiver 110 may be configured to receive light collected by the probe 102.

The subsystem may include a computing device 112. The computing device may include one or more processors 113, memory 114, instructions 115, data 116, and one or more modules 117.

The one or more processors 113 may be any conventional processors, such as commercially available microprocessors. Alternatively, the one or more processors may be a dedicated device such as an application specific integrated circuit (ASIC) or other hardware-based processor. Although FIG. 1 functionally illustrates the processor, memory, and other elements of device 110 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of device 112. Accordingly, references to a processor or computing device will be understood to include references to a collection of processors or computing devices or memories that may or may not operate in parallel.

Memory 114 may store information that is accessible by the processors, including instructions 115 that may be executed by the processors 113, and data 116. The memory 114 may be a type of memory operative to store information accessible by the processors 113, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions 101 and data 119 are stored on different types of media.

Memory 114 may be retrieved, stored or modified by processors 113 in accordance with the instructions 115. For instance, although the present disclosure is not limited by a particular data structure, the data 115 may be stored in computer registers, in a relational database as a table having a plurality of different fields and records, XML documents, or flat files. The data 115 may also be formatted in a computer-readable format such as, but not limited to, binary values, ASCII or Unicode. By further way of example only, the data 115 may be stored as bitmaps comprised of pixels that are stored in compressed or uncompressed, or various image formats (e.g., JPEG), vector-based formats (e.g., SVG) or computer instructions for drawing graphics. Moreover, the data 115 may comprise information sufficient to identify the relevant information, such as numbers, descriptive text, proprietary codes, pointers, references to data stored in other memories (including other network locations) or information that is used by a function to calculate the relevant data. Memory 114 can also contain or store a set of training data, such as OCT images, to be used in conjunction with a machine learning model to train the machine learning model to analyze OCT images not contained in the set of training data.

The instructions 115 can be any set of instructions to be executed directly, such as machine code, or indirectly, such as scripts, by the processor 113. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. Functions, methods and routines of the instructions are explained in more detail below.

The modules 117 may include a display module. In some examples further types of modules may be included, such as modules for computing other vessel characteristics. According to some examples, the modules may include an image data processing pipeline or component modules thereof. The image processing pipeline may be used to transform collected OCT data into two-dimensional (“2D”) and/or three-dimensional (“3D”) views and/or representations of blood vessels, stents, and/or detected regions. The modules 117 can also contain image recognition and image processing modules to identify and classify one or more elements of an image.

The modules 117 may include a machine learning module. The machine learning module can contain machine learning algorithms and machine learning models, including neural networks and neural nets. The machine learning module can contain machine learning models which can be trained using a set of training data. In some examples and without limitation, the machine learning module or machine learning algorithms can contain or be made of any combination of a convolution neural network, a perceptron network, a radial basis network, a deep feed forward network, a recurrent neural network, an auto encoder network, a gated recurrent unit network, a deep convolution network, a deconvolution network, or a support vector machine network. In some examples, the machine learning algorithms or machine learning models can be configured to take as an input a medical diagnostic image, such as an OCT image, and provide as an output a probability that the image belongs to a particular classification or category.

The subsystem 108 may include a display 118 for outputting content to a user. As shown, the display 118 is separate from computing device 112 however, according to some examples, display 118 may be part computing device 112. The display 118 may output image data relating to one or more features detected in the blood vessel. For example, the output may include, without limitation, cross-sectional scan data, longitudinal scans, diameter graphs, image masks, etc. The output may further include lesions and visual indicators of vessel characteristics or lesion characteristics, such as computed pressure values, vessel size and shape, or the like. The output may further include information related to the OCT images collected, such as the regions where the OCT images obtained were not “clear” or summary information about the OCT scan, such as the overall quality of the scan. The display 118 may identify features with text, arrows, color coding, highlighting, contour lines, or other suitable human or machine readable indicia.

According to some examples the display 118 may include a graphic user interface (“GUI”). According to other examples, a user may interact with the computing device 112 and thereby cause particular content to be output on the display 118 using other forms of input, such as a mouse, keyboard, trackpad, microphone, gesture sensors, or any other type of user input device. One or more steps may be performed automatically or without user input to navigate images, input information, select and/or interact with an input, etc. The display 118 and input device, along with computing device 112, may allow for transition between different stages in a workflow, different viewing modes, etc. For example, the user may select a segment of vessel for viewing an OCT image and associated analysis of the OCT image, such as whether the image is considered to be acceptable/clear or unacceptable/blocked, as further explained below.

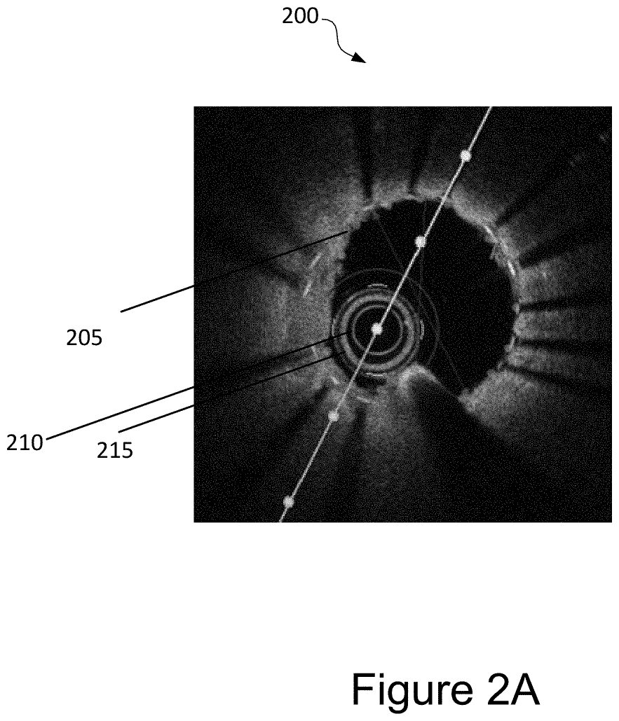

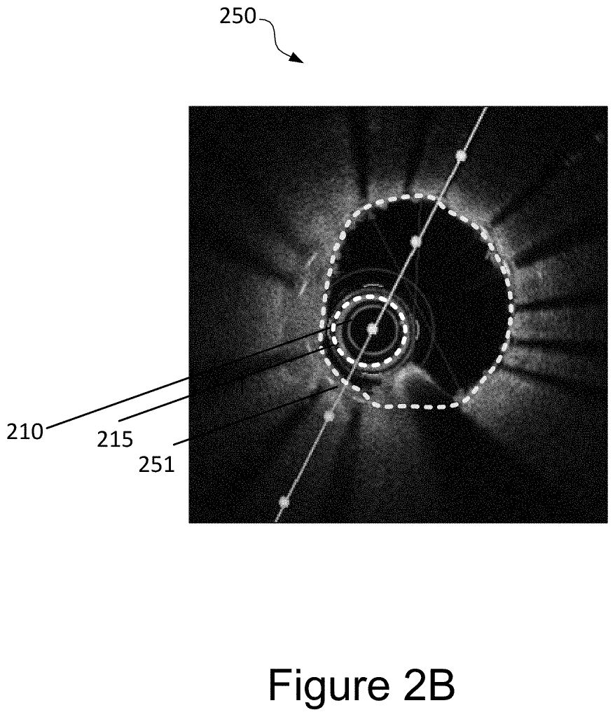

FIG. 2A illustrates “clear” OCT images. Illustrated in FIG. 2A is a clear OCT image 200. OCT image 200 is a cross sectional representation of a portion of vascular tissue. OCT images can be inhomogeneous, vary in degree, intensity, and shape, and contain artifacts, such as bright concentric rings or bright structure emerging from the guidewire. Illustrated in image 200 is lumen 205 as well as the centrally located OCT guidewire 210 contained within the perimeter of OCT guide catheter 215. OCT image 200 is clear as there is no obstruction to viewing the lumen or artifacts in the image other than the guide catheter. In OCT image 200, the contour of the lumen is visible in the image and the presence of blood, if any, is minimal or under a pre-determined threshold. OCT image 200 is thus “clear.”

FIG. 2B illustrates an annotated version of the image 200, annotated clear OCT image 250. For reference, OCT guidewire 210 and OCT guide catheter 215 are labeled in FIG. 2B. Annotated clear image 250 is an annotated or marked version of clear OCT image 200, which marks the lumen with lumen annotation 251. Similar to lumen annotation 251 the guide catheter can also be annotated, as depicted in a dashed line in FIG. 2B. In some examples, particular sets of annotations can be used to train a machine learning model while other annotations are ignored or not used for training. For example, as it may be expected that the guide catheter is present in all OCT images, it may not be used in training a machine learning model or later used in categorizing a new image. Image 250 can be categorized as “clear” as the lumen is largely visible and there is no major obstruction to viewing the lumen.

Image 250 can also be associated with a tag, metadata, or placed into a category, such as “clear” to indicate that the image is considered clear when used for training a machine learning model. The machine learning model can be configured to perform classification of new images. Classification is a technique for determining the class to which the dependent variable belongs based on one or more independent variables. Classification thus takes as an input one or more independent variables and outputs a classification or probability related to a classification. For example, image 250 can be part of a set of machine learning training data which is used to train a machine learning model to classify new images. By using the categorization of images within the set of data used to train the machine learning model, including images such as image 250 and its associated category, a machine learning algorithm can be trained to evaluate which features or combination of features lead to a particular image being categorized as “clear” or be categorized in a different category.

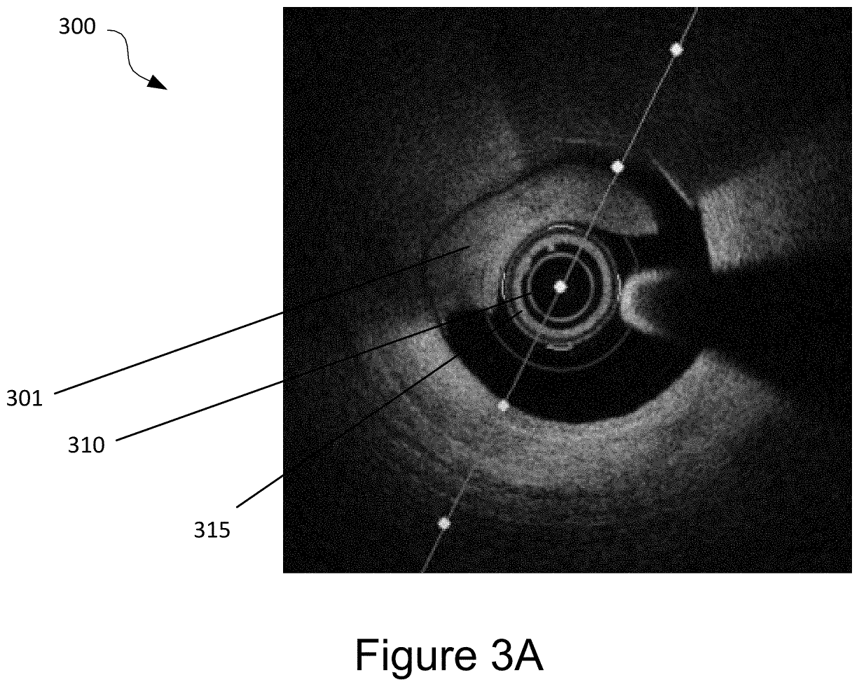

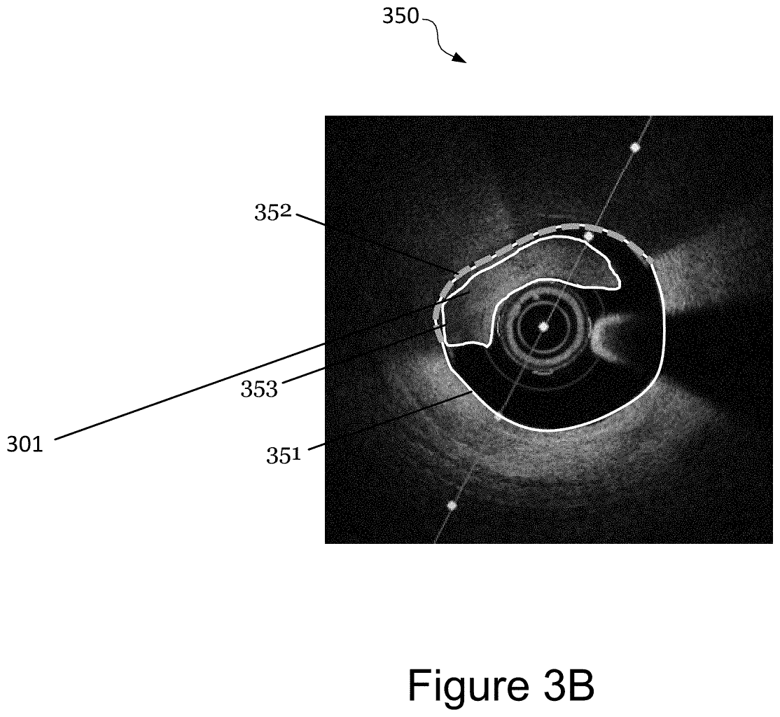

FIG. 3A illustrates “blocked” OCT images. Illustrated in FIG. 3 is a blocked OCT image 300. As can be seen in OCT image 300, a portion of the image is blocked by blood 301 in the upper left portion of the image and surrounding the centralized guide catheter 315 and guide wire 310.

In some examples, a degree of blockage to be considered “blocked” or “unacceptable” may be configurable by a user or preset during manufacture. By way of example only, images in which 25% or more of the lumen is blocked by blood can be considered to be “blocked” images.

FIG. 3B illustrates an annotated “blocked” OCT image. Annotated blocked OCT image 350 illustrates an annotated version of blocked OCT image 300. Annotation 351 (solid circular line) illustrates the lumen portion, annotation 352 illustrates the portion of the lumen blocked by blood 301, and annotation 353 (upper left convex closed shape) illustrates the portion of the OCT image which is blood. Similar to clear annotated image 250, blocked annotated image 350 can also be associated with a tag, metadata, or be placed into a category, such as “unclear,” “blocked,” or “blood” to indicate that the image is not acceptable, or is considered to be unclear, when used for training in a machine learning model.

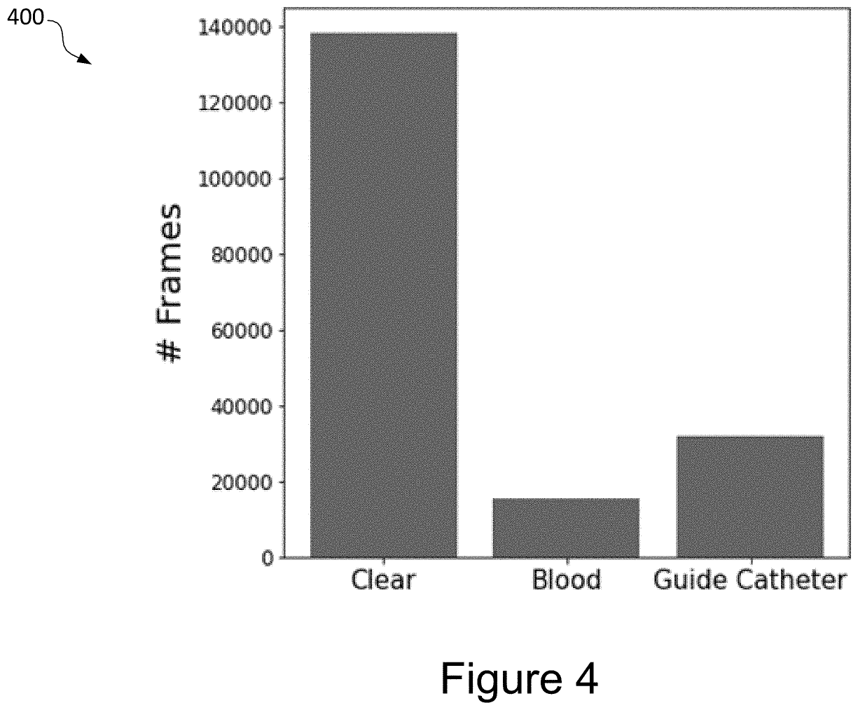

FIG. 4 illustrates a histogram 400 associated with a training set of data. The training data can include, for example, OCT images having varying degrees of clarity or blockage, such as those described above in connection with FIGS. 2 and 3. The training set of data may further include additional information, such as annotations, metadata, measurements, or other information corresponding to the OCT images that may be used to classify the OCT images. The training set of data can include any number of images, with higher numbers of images providing for increased accuracy of the machine learning model. For example, hundreds or thousands of OCT images can be used, which can be obtained from various OCT pullbacks or other OCT measurements. The relative proportion of the images which have been categorized or consist of a guide catheter, are blocked due to blood, or are clear are indicated or visible in histogram 400. The relative proportion of images can be tuned or adjusted to tune the training of the trained machine learning model. The training set of data can be adjusted to have an appropriate proportion of these the various categories to ensure proper training. For example, if the training set is “unbalanced,” such as, for example, by containing a larger number of images which are clear, the machine learning model may not be sufficiently trained to distinguish features which cause an image to not be “clear” and may be biased to artificially boost performance simply by classifying most of the images as “clear.” By using a more “balanced” training set, this issue can be avoided.

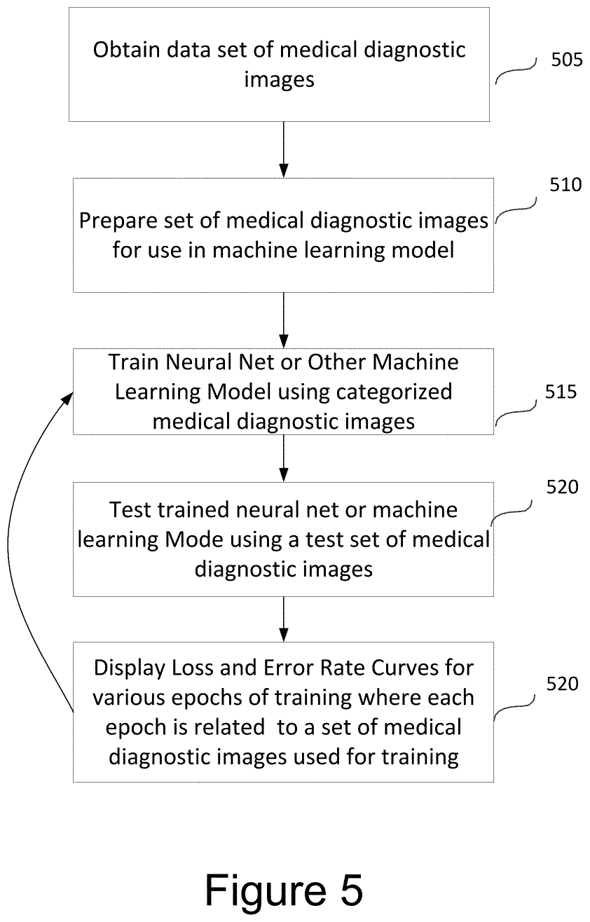

FIG. 5 illustrates a flowchart of a method 500. Method 500 can be used to train a neural net, a neural network, or other machine learning model. Neural networks or neural nets can consist of a collection of simulated neurons. Training of a neural network can include weighing various connections between neurons or connections of the neural network. Training of the neural network can occur in epochs over which an error associated with the network can be observed until the error sufficiently converges. In some examples and without limitation, the neural net or neural network can be a convolution neural network, a perceptron network, a radial basis network, a deep feed forward network, a recurrent neural network, an auto encoder network, a gated recurrent unit network, a deep convolution network, a deconvolution network, a support vector machine network, or any combination of these or other types of networks.

At block 505, a set of medical diagnostic images can be obtained. In some examples, the set of medical diagnostic images can be obtained from an OCT pullback or other intravascular imaging technique. In other examples, the set of medical diagnostic images can be randomized or taken from various samples, specimens, or vascular tissue to provide a large sample size of images. This set of medical diagnostic images can be similar to OCT image 200 or OCT image 300.

At block 510, the set of medical diagnostic images can be prepared to be used as a dataset for training a machine learning model. At this block one or more techniques can be used to prepare the set of medical diagnostic images to be used as training data.

For example, the medical diagnostic images can be annotated. Portions of each medical diagnostic image from the set of medical diagnostic images can be annotated to form images similar to, for example, annotated clear OCT image 250 or annotated blocked OCT image. For example, each image can have portions of the image annotated with “clear” or “blood” to represent portions of the image which represent an image. For example, the set of medical diagnostic images, which can be used for training, can be annotated or categorized to create images similar to annotated clear OCT image 250 and annotated blocked OCT image 350. In other examples, the annotations can be digitally drawn on the images to identify portions of the image which correspond to particular features, such as lumen, blood, or guide catheter. In some examples, the annotation data can be represented as a portion of the image or a set of pixels.

The medical diagnostic images can also be categorized or separated into categories. In some examples, the categorization can take place through a human operator. For example, the medical diagnostic images can be classified between the values of a binary set, such as [unacceptable, acceptable], [unclear, clear], [blocked, unblocked] or [not useful, useful]. In some examples, non-binary classifications can be used, such as a set of classifications which can indicate a percentage of blockage, e.g. [0% blocked, 20% blocked, 40% blocked, 60% blocked, 80% blocked, or 100% blocked]. Each medical diagnostic image may be placed into a category most closely representing the medical diagnostic image.

In some examples, multiple types of classifications can be used on the medical diagnostic image. The medical diagnostic images may be associated with multiple sets of categories. For example, if a medical diagnostic image has a stent and likely blood blocked, the classification for the image may be <stent, blocked>. Another example may be if the frame contains a guide catheter or not, and the classification for the image may be <catheter, blocked>. Multiple classifications can be used collectively during the training of machine learning models or classification of data.

In some examples, the set of training data can be pruned or adjusted to contain a desired distribution of blocked and clear images

The set of medical diagnostic images can be reworked, manipulated, modified, corrected, or generalized prior to use in training. The manipulation of the medical diagnostic images allows for the training of the machine learning model to be balanced with respect to one or more characteristics, as opposed to being overfit for particular characteristics. For example, the medical diagnostic images can be resized, transformed using random Fourier series, flipped in polar coordinates, rotated randomly, adjusted for contrast, brightness, intensity, noise, grayscale, scale, or have other adjustments or alterations applied to them. In other examples, any linear mapping represented by a matrix can be applied to the OCT images. Underfitting can occur when a model is too simple, such as with two few features, and does not accurately represent the complexity needed to categorize or analyze new images. Overfitting occurs when a trained model is not sufficiently generalized to solve the general problem intended to be represented by the training set of data. For example, when a trained model more accurately categorizes images within a training set of data, but has lower accuracy on a test set of data, the trained model can be said to be overfit. Thus, for example if all images are of one orientation or have a particular contrast, the model may become overfit and not be able to accurately categorize images which have a different contrast ratio or are differently oriented.

At block 515, a neural network, neural net, or machine learning model can be trained using the categorized data set. In some examples, training of the machine learning model can proceed in epochs until an error associated with the machine learning model sufficiently converges or stabilizes. In some examples, the neural network is trained to classify images, such as in a binary set of images. For example, the neural network can be trained based on the set of training data which includes clear and blocked images and be trained to output either “clear” or “blocked” as an output.

At block 520 the trained neural net, neural network, or machine learning model can be tested. In some examples, the neural network can be tested based on images which were not used for training the network and whose classification is otherwise known. In some examples, images which are considered to be “edge cases” upon being analyzed, such as those which cannot clearly be classified, can be used to retrain the neural network after manual classification of the images. For example, if the determination of whether a particular image depicts a blood-filled vessel cross-section or a clear vessel cross-section has low confidence, that particular image can be saved for analysis by a human operator. Once categorized by the human operator, the image can be added to the set of data used to train the machine learning model and the model can be updated with the new edge case image.

At block 525, learning curves, such as loss or error rate curves for various epochs of training the machine learning model can be displayed. In some examples, each epoch can be related to a unique set of OCT images which are used for training the machine learning model. Learning curves can be used to evaluate the effect of each update during training and measuring aspects and plotting the performance of the model during each epoch or update can provide information about the characteristics and performance of the trained model. In some examples, a model can be selected such that the model has minimum validation loss, so that the validation loss training curve is most important. Blocks 515 and 520 can be repeated until the machine learning model is sufficiently trained and the trained model has desired performance characteristics. As one example, the computational time or computational intensity of the trained model can be a performance characteristic which is below a certain threshold.

The model can be saved at the epoch which contains the lowest validation loss, and this model, with its trained characteristics, can be used to evaluate performance metrics on a test set which may not have been used in training. If the performance of such a model passes a threshold, the model can be considered to be sufficiently trained. Other characteristics related to the machine learning model can also be studied. For example, a receiver operating characteristic curve or a confusion matrix can be used to evaluate the performance of the trained machine learning model.

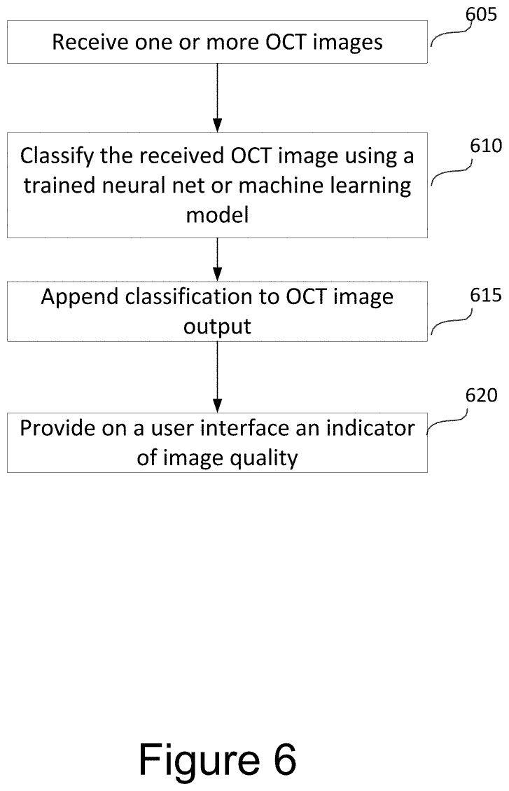

FIG. 6 provides a flowchart illustrating a method 600 of classifying images in a medical diagnostic procedure. Method 600 can be used to characterize an OCT image, or a series of OCT images. For example, method 600 can be used to characterize a series of OCT images which are associated with an OCT pullback in which OCT images corresponding to a particular length of vascular tissue, such as an artery, are obtained. Such characterization may be used to indicate to a physician in real time whether images having a predefined threshold of quality were obtained. In this regard, if the image quality for an OCT pullback was not sufficient, the physician can perform another pullback within the same medical procedure when the OCT probe and catheter are still within the patient's vessel, as opposed to requiring a follow-up procedure where the OCT catheter and probe would need to be reinserted.

At block 605, one or more unclassified OCT images can be received. The received OCT images can be associated with a particular location within a vascular tissue and this location can later be used to create various representations of the data obtained during the OCT.

At block 610, the received OCT image can be analyzed or classified using a trained neural network, trained neural net, or trained machine learning model. The trained neural network, trained neural net, or trained machine learning model has been trained and tuned to identify various features, such as lumen or blood, from the training set of data. These parameters can be identified using image or object recognition techniques. In other examples, a set of characteristics can be gleaned from the image or image data which may be known or hidden variables during the training of the machine learning model or neural network. For example, the relative color, contrast, or roundness of elements of the image may be known variables. Other hidden variables can be derived during the training process and may not be directly identified but are related to a provided image. Other variables can be related to the image metadata, such as which OCT system took the image. In other examples, the trained neural network can have weightings between the various neurons or connections of the network based on the training of the network. These weighted connections can take the input image and weigh various parts of the image, or features contained within the image, to produce a final result, such as a probability or classification. In some examples, the training can be considered to be supervised as each input image has a manual annotation associated with it.

The trained neural network, trained neural net, or trained machine learning model can take as an input the OCT image and provide as an output a classification of the image. For example, the output can be whether the image is “clear” or “blocked.” In some examples, the neural network, neural network, or machine learning model can provide a probability associated with the received OCT image, such as whether the OCT image is “clear” or “blocked.”

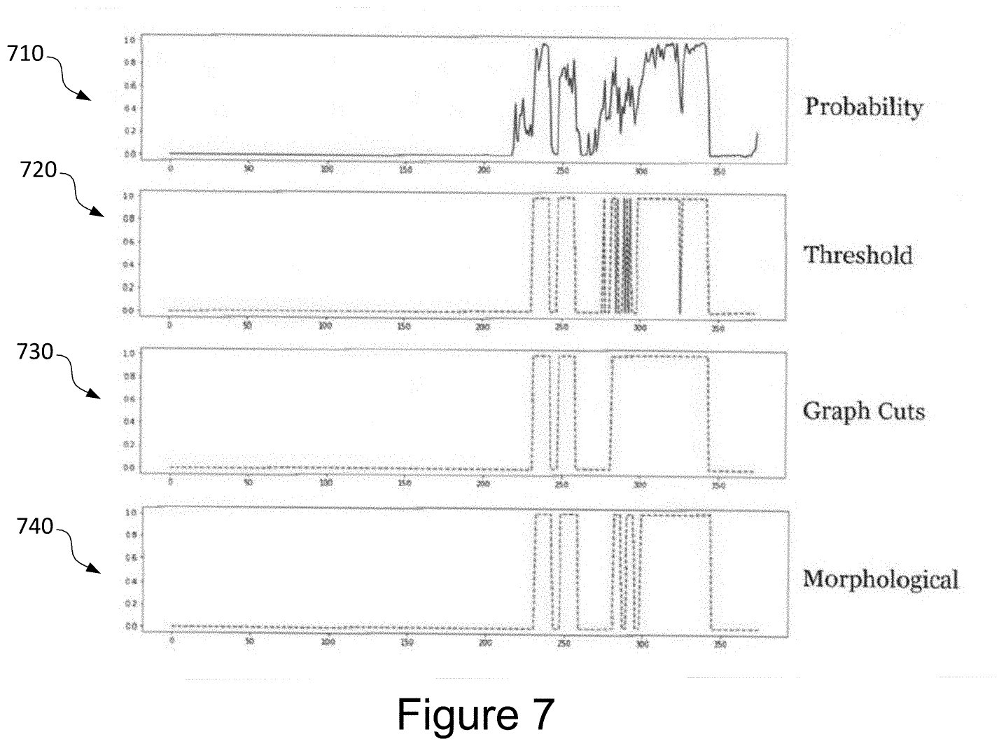

In some examples, such as those described with respect to FIG. 7, additional methods can be used to classify or group a sequence of OCT images.

In other examples, multiple neural networks or machine learning models can be used to process the OCT image. For example, any arbitrary number of models can be used and the probability outcomes of the models can be averaged to provide a more robust prediction or classification. The use of multiple models can optionally be used when a particular image is difficult to classify or is an edge case where one model is unable to clearly classify the outcome of the OCT image.

At block 615, the output received from block 610 can be appended or otherwise associated with the received OCT image. This information can be used when displaying the OCT images to a user.

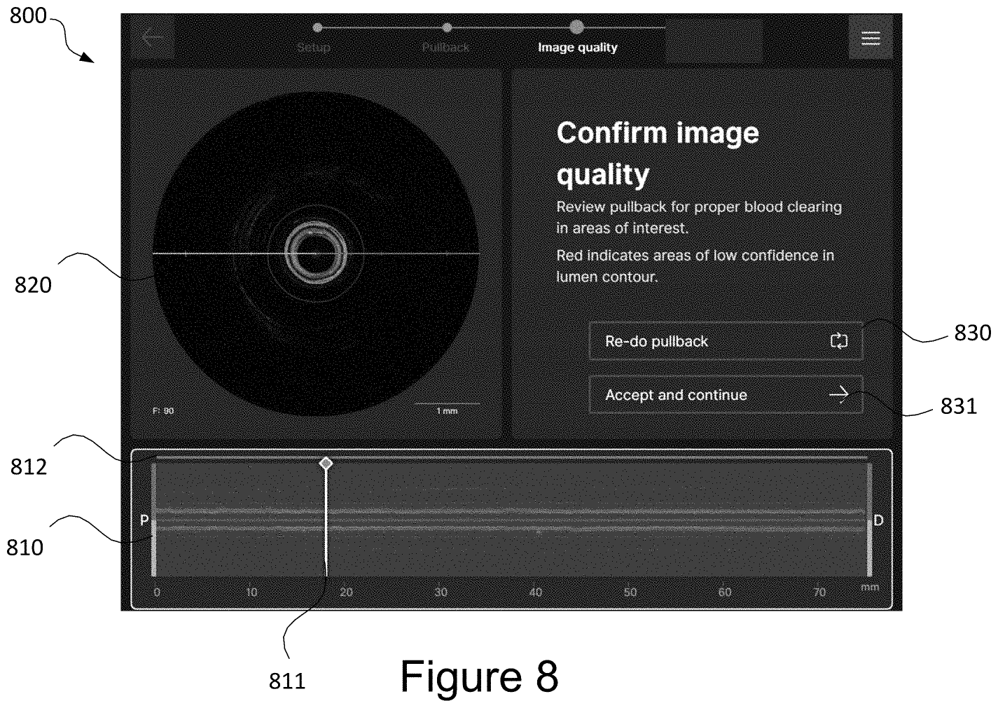

At block 620, information about the OCT images and/or information about the OCT image quality can be provided to a user on a user interface. Additional examples of user interfaces are given with respect to FIG. 8. For example, the information can be displayed along with each OCT image or a summary of an OCT scan or OCT pullback. In some examples, a longitudinal view of a vessel, such as shown in FIG. 8, can be created from the combination of OCT images and information about which portions of the vessel were not imaged due to “blocked” images can be displayed alongside the longitudinal view.

In other examples, summary information about the scan can be provided for display on a display to a user. The summary information can contain information such as the number of frames or OCT images which were considered blocked or the overall percentage of OCT images which were considered clear and identify areas where a cluster of OCT images were blocked. The summary information. In other examples, the summary information or notification can provide additional information as to why a particular frame was blocked, such as the OCT pullback being performed too quickly.

FIG. 7 illustrates aspects of techniques which can be used to classify or group a sequence of OCT images from a probability. Illustrated in FIG. 7 is graph 710, representing the probability that a particular image is “clear” or “blocked” on a scale from 0 to 1. Graph 710 is a raw probability value which can be obtained from a trained machine learning model or a neural network. A probability of 0 implies that the image is considered to be completely clear while a probability of 1 implies that the image is considered to be blocked. Values between 0 and 1 represent the likelihood that an image is clear or blocked. The horizontal x-axis in graph 710 can represent the frame number of a sequence of OCT images or OCT frames, such as those obtained during an OCT pullback. The horizontal x-axis can also be related to a proximal or distal location of vascular tissue which was imaged to create the OCT image.

Graph 720 illustrates the use of a “threshold” technique to classify the probability distribution of graph 710 into a binary classification. In a threshold technique, OCT images with probability values above a certain threshold can be considered to be “blocked” while those with probability values under the same threshold can be considered to be “clear.” Thus, graph 710 can be used as an input and graph 720 can be obtained as an output.

Graph 730 illustrates the use of graph cut techniques to classify the probability distribution of graph 710. For example, graph cut algorithms can be used to classify the probability as either “clear” or “blocked.”

Graph 740 illustrates the use of morphological techniques to classify the probability distribution of graph 710. Morphological techniques apply a structuring element to an input image, creating an output image of the same size. In a morphological operation, the value of each pixel in the output image is based on a comparison of the corresponding pixel in the input image with its neighbors. The probability values of graph 710 can be compared in this manner to create graph 740.

FIG. 8 illustrates an example user interface 800 illustrating aspects of lumen contour confidence and image quality. User interface 800 illustrates a linear representation of a series of OCT images in component 810 with the horizontal axis indicating the location or depth within a vascular tissue. Indicator 811 within component 810 can represent the current location within a vascular tissue or depth within a vascular tissue being represented by OCT image 820. Indicator 812 can be a colored indicator which corresponds to the horizontal axis. Indicator 812 can be colored, such as with red, to represent the probability or confidence that an OCT image associated with that location is “blocked” or “clear.” In some examples, a white or translucent overlay may exist on portions of the image corresponding to indicator 812 to further indicate that the area is of low confidence. Image 820 can be the OCT image at the location represented by indicator 812. Image 820 may also contain coloring or other indicator to indicate portions of a lumen which are areas of low confidence. User interface 800 can also contain options to re-perform the OCT pullbacks or accept the results of the OCT pullback.

In some examples, additional meta-data related to image 820 may be displayed on user interface 800. For example, if additional information about the image is available, such as for example, resolution of the image, the wavelength of the image used, the granularity, the suspected diameter of the OCT frame, or other meta-data related to the OCT pullback which may assist a physician in evaluating the OCT frame.

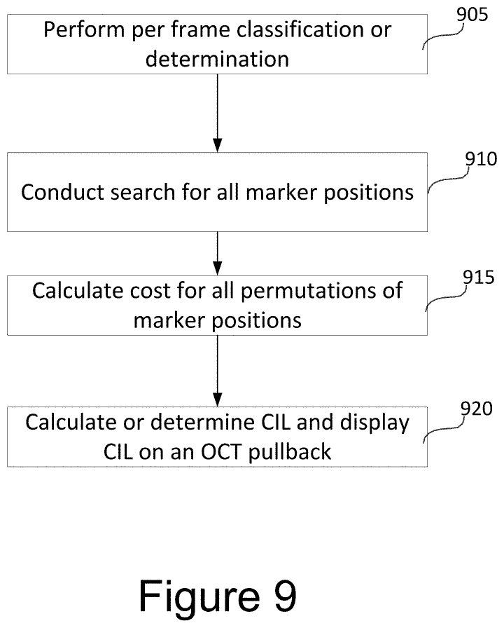

As shown in FIG. 8, the interface may further provide a prompt to the physician in response to the notification or other information relating to the machine learning evaluation of the image. For example, the prompt may provide the physician with a choice whether to accept the collected image and continue to a next step of a procedure, or to repeat the image collection steps, such as by performing another OCT pullback. For example, user interface 800 may contain prompt 830 which can enable an OCT pullback to be repeated. Upon selecting or interacting with prompt 830, computing devices can cause OCT equipment to be configured to receive additional OCT frames. Interface 800 may also contain prompt 831 which allows for the results of the OCT to be accepted. Upon interacting with prompt 831, additional OCT frames would not be accepted. In addition, as further explained with reference to FIGS. 9 to 11, user interface 800 may display a clear image length (CIL) of an OCT pullback. In some examples, user interface 800 may suggest or require that an OCT pullback be performed again when the CIL is smaller than a predetermined length.

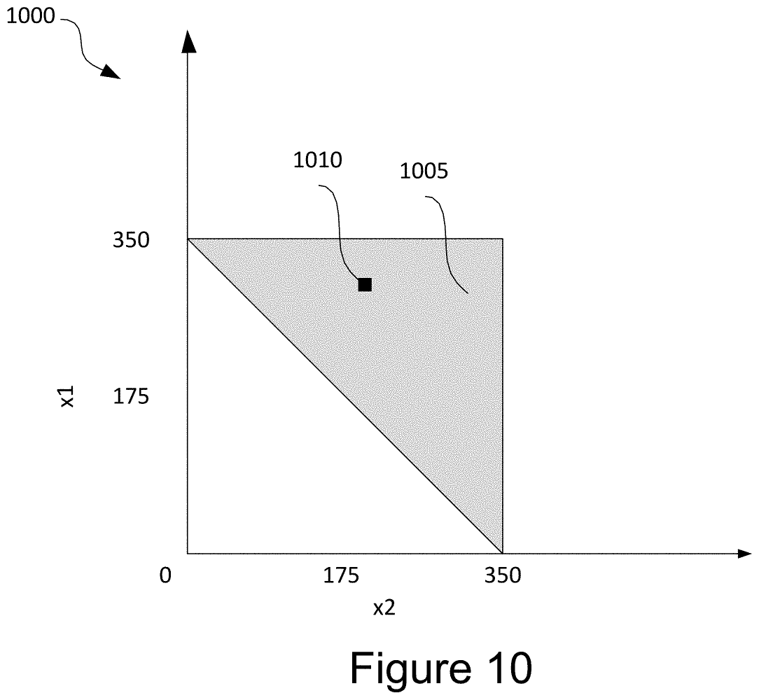

FIG. 9 illustrates method 900. Method 900 can be used to produce or calculate a clear image length (CIL) of an OCT pullback. A clear image length or CIL can be an indication or information related to a contiguous section of an OCT pullback which is not obstructed or determined to be clear, such as for example not being blocked by blood or being considered a blood frame. A CIL vector score for a pullback of “n” frames can be calculated with a value between 0 and n. A score of 0 can represent a complete mismatch while a score of n implies a complete match. An example of a CIL vector score is given with reference to FIG. 10. A match can refer to a classification which matches a CIL classification. In some examples, within a CIL classification, everything in an “exclusion zone” can be a 0 while everything outside an exclusion zone can be a 1. If the CIL classification matches the per frame classification, a “1” can be added to a score, and if they do not match, a 0 can be added to a score. A CIL with the highest score can be selected.

At block 905, for a given OCT pullback, a per-frame quality assurance classification can be performed on each OCT image within a pullback. In some examples, a binary classifier can be used which results in a 0 or 1 score for each OCT frame. In other examples, such as through using assembling techniques, a value ranging between 0 to 1 can be generated for each OCT frame.

At block 910, an exhaustive search for marker positions, such as marker x1 and marker x2, is performed. In some examples, x1 can correspond to a blood marker and x2 as a clear marker. For example, with reference to FIG. 8, marker 840 and marker 841 can correspond to x1 and x2 respectively. By varying marker 840 and marker 841, all combinations can be evaluated. After performing the search for each position, a permutation for each x1 and x2 position can be calculated such that x2>x1, leading to roughly a computational complexity of (N\hat{ }2)/2.

At block 915, for each permutation, a cost related to that permutation can be calculated and a global optimal or maximum for the cost be determined. In some examples, the cost can be computed by summing the number of matches between an auto image quality vector score vector and a corresponding CIL score vector. An example of a computed score is given with reference to FIG. 10. The maximum point on FIG. 10 can correspond to the longest or maximal CIL within an OCT pullback. The position of the max value of this cost matrix is the resulting optimal x1 and x2 positions for the CIL. In some examples, the CIL is the “best” possible contiguous range of non-blood frames but may still contain some blood frames. In some examples, the CIL can be a measure of the position of the bolus of contrast in the pullback. In other examples, it is possible to have some blood frames within this bolus due to side branches and mixing of the bolus with blood.

In some examples, the CIL can be computed automatically during an OCT pullback. In some examples, information related to the CIL can be used by downstream algorithms to avoid processing images which are obstructed by blood to improve the performance of OCT imaging systems and increase computational efficiency of the OCT system.

At block 920, based on the optimal or maximal CIL calculated, a CIL indicator can be plotted on an OCT image. For example, the CIL can be plotted between dashed colored lines. Outside the CIL, if there are OCT frames which are detected or classified as “blood” frames, those frames can be overlaid in a transparent red color to indicate that the frame is a “blood” frame. Within the CIL, if there are frames which are detected as blood, those frames can be visually smoothed over and displayed as transparent red.

FIG. 10 illustrates an example CIL cost matrix 1000. Cost matrix 1000 can be a top-right matrix as the values for x2>=x1. Region 1005 can be the region of allowed or feasible values of x1 and x2. Also illustrated on cost matrix 1000 is point 1010, a maximum value, discussed with reference to block 910. Point 1010 can be calculated from the values of x1 and x2 within the region 1005. Point 1010 can correspond to a maximum value of a cost function. In some examples, region 1005 can be colored in a gradient to illustrate intensities and costs in a 2-D format, and point 1010 can be chosen to be the maximum value of a cost function.

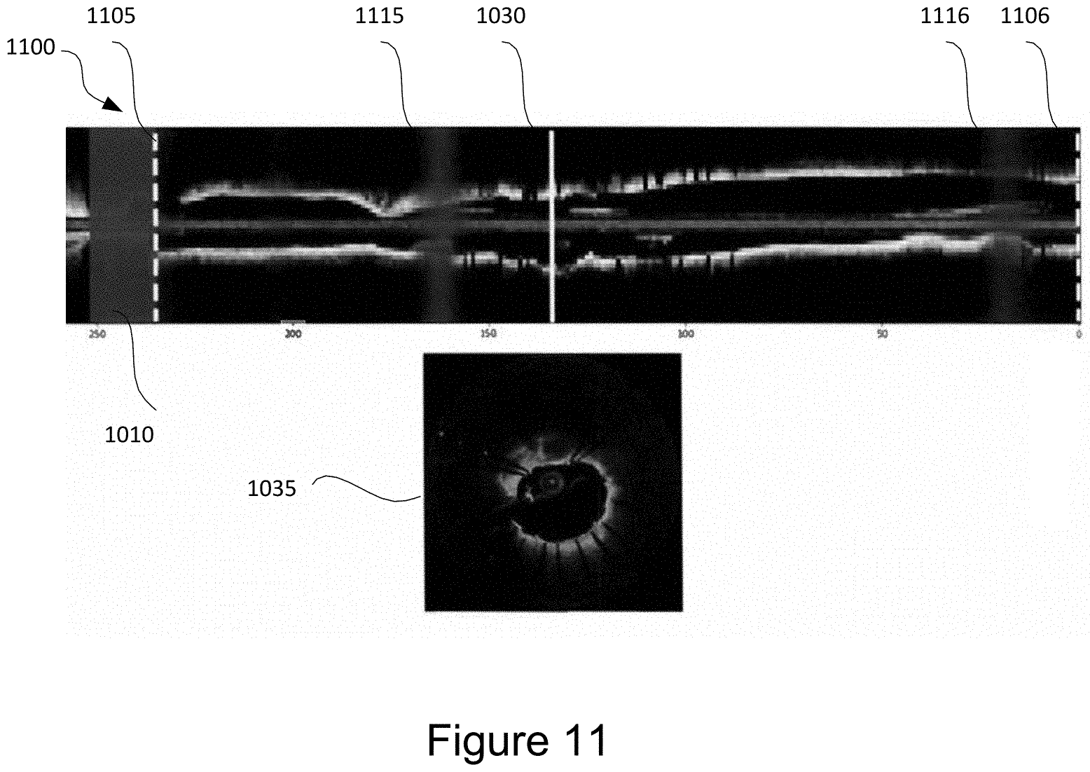

FIG. 11 illustrates an example OCT pullback 1100 with a CIL incorporated into the OCT pullback. A CIL incorporated into an OCT pullback can also be seen with respect to FIG. 8. For example, with reference to FIG. 8, marker 840 and marker 841 can correspond to x1 and x2 respectively. The CIL can be to the length between marker 840 and marker 841.

OCT pull back 1100 can be displayed on a graphical user interface or user interface, such as user interface 800 (FIG. 8). The horizontal axis of OCT pullback 1100 can indicate an OCT frame number, a location within a vascular tissue, or depth within a vascular tissue. Illustrated in FIG. 11 are various indicia included on OCT pullback 1100. Dashed line 1105 and dashed line 1106 can indicate the boundaries of the CIL. Illustrated within the boundaries of the CIL are blood region 1115 and blood region 1116, indicated with a blurry area. Region 1120 to the left of dashed line 1105 indicates an area outside the boundaries of CIL. In some examples, region 1120 can contain an overlaid translucent, transparent, or semi-transparent image to provide a visual indication to a user that the area is outside the CIL. Location indicator 1130 can indicate the location within OCT pullback 1100, which corresponds to OCT frame 1135.

The technology can provide a real time or near real time notification containing information related to image quality as an OCT procedure is being performed based on the trained machine learning model or trained neural network. For example, the notification may be an icon, text, audible indication, or other form of notification that alerts a physician as to a classification made by the machine learning model. For example, the notification may identify the image as “clear” or “blocked.” According to some examples, the notification may include a quantification of how much blood blockage is occluding the vessel in a particular image frame or vessel segment. This allows physicians to have an immediate indication of whether the data and images being obtained are sufficiently clear for diagnostic or other purposes and does not require manual checking of hundreds or thousands of images after the procedure is done. As it may not be practical for all OCT images to be manually checked, the technology prevents improper interpretation of OCT scans which are improper or not sufficiently clear.

In addition, as the analysis can be done in real time, a notification or alert related to the OCT images can indicate which portions of an OCT scan or OCT pullback were not of sufficiently clear quality (or were blocked) and allow those portions of the OCT scan or OCT pullback to be performed. This allows a physician to perform another OCT scan or OCT pullback of those portions which were not sufficiently clear while the OCT device is still in situ and avoids the need for the patient to return for another procedure. Further, the computing device can replace those portions of the scan which were considered deficient or blocked with the new set of OCT images and “stitch” or combine the images to provide a singular longitudinal view of a vessel obtained in an OCT pullback.

In addition, identification of portions of the OCT scan or OCT pullback which are not considered to be acceptable or clear can be evaluated by a physician to determine if the physician is interested in the region corresponding to the blocked OCT images.

Further, a summary of the OCT scan or OCT pullback can be provided to a user. For example, the summary information can include information about the overall percentage or number of frames which are considered acceptable, whether a second scan is likely to improve the percentage of frames. In other examples, the summary information or notification can provide additional information as to why a particular frame was blocked, such as the OCT pullback being performed too quickly or blood not being displaced.

While in some examples a user or physician may define whether an image is clear or blocked, such as by setting thresholds used in the detection of image quality, in other examples a confidence level of a computational task may be used to determine whether the image is sufficiently clear or not. For example, a task-based image quality assessment method is described herein. The task-based image quality assessment method may be beneficial in that it does not require human operators to select high- and low-quality image frames to train a prediction model. Rather, image quality is determined by the confidence level of the task being achieved. The image quality assurance method can accommodate evolution of the technology used in the computational task. For example, when technologies for accomplishing tasks advance further and further, the image quality assurance results will be evolved together to reflect the image quality more realistically. The task-based quality assurance can help users to keep as many OCT frames as possible, while ensuring the clinical usability of these frames.

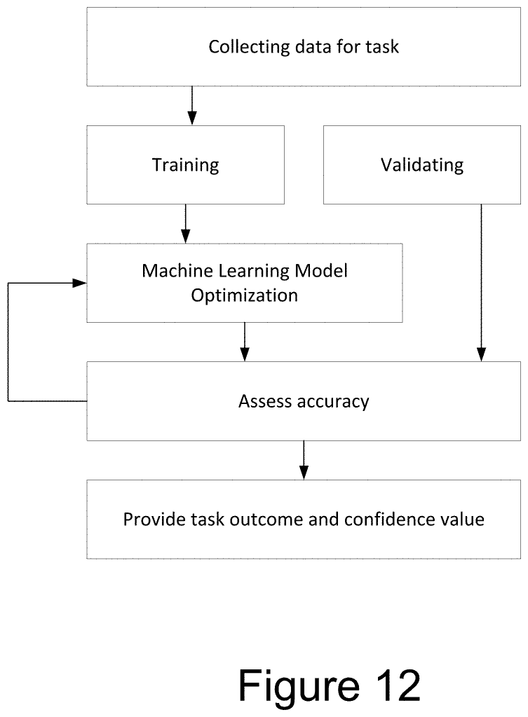

FIG. 12 is a flow diagram illustrating an example method 1200 of assuring image quality using a machine-learning task-based approach. The task may be any of a variety of tasks, such as lumen contour detection, calcium detection, or detection of any other characteristic. Lumen contour detection may include, for example, geometric measurements, detection of vessel walls or boundaries, detection of holes or openings, detection of curves, etc. Such detection may be used in assessing severity of vessel narrowing, identifying sidebranches, identifying stent struts, identifying plaque, EEL or other media, or other types of vessel evaluation.

In block 1210, data is collected for the task. The data may be, for example, intravascular images, such as OCT images, ultrasound images, near-infrared spectroscopy (NIRS), micro-OCT, images, or any other type of images. In some examples, the data may also include information such as patient information, image capture information (e.g., date, time, image capture device, operator, etc.), or any other type of information. The data may be collected using one or more imaging probes from one or more patients. According to some examples, the data may be retrieved from a database storing a plurality of images captured from a multitude of patients over a span of time. In some examples, the data may be presented in a polar coordinate system. According to some examples, the data may be manually annotated, such as to indicate the presence and location of lumen contours where the task is to identify lumen contours. Moreover, the data may be split into a first subset used for training and a second subset used for validation.

In block 1220, a machine learning model is trained using the collected data. The machine learning model may be configured in accordance with the task. For example, the model may be configured to detect lumen contours. Training the model may include, for example, inputting collected data that matches the task. For lumen detection, training the model may include inputting images that depict lumen contours.

In block 1230, the machine learning model is optimized based on the training data. In the example of lumen contour detection task, the model input may be a series of gray level OCT images which can be in the form of a 3D patch. A 3D patch is a stack of consecutive OCT images, where the size of the stack depends on the computational resource, such as the memory of a graphical processing unit (GPU). The model output during training may include a binary mask of each corresponding stack manually annotated by human operators. Manual annotation on 3D patches is time consuming, and therefore a data augmentation preprocessing step may be included before optimizing the machine learning model. The data augmentation may be performed on the annotated data with variations, such as random rotation, cropping, flipping, and geometric deformation of the 3D patches of both OCT images and annotations, such that a sufficient training dataset is produced. The data augmentation process can vary by the types of tasks. Once the data augmentation step is determined, a loss function and optimizer are specified as cross-entropy and Adam optimizer. Similarly, the loss and optimizer (and other hyperparameters in the training process) may vary by the types of tasks and image data. The machine learning model is optimized until the loss function value that measures the discrepancy of the model computational output and the expected output is minimized within a given number of iterations, or epoch.

In block 1240, the validation set of data may be used to assess the accuracy of the machine learning model. For example, the machine learning model may be executed using the validation data and it may be determined whether the machine learning model produced the expected result for the validation data. For example, an annotated validation image and an output of the machine learning model may be compared to determine a degree of overlap between the annotation validation image and the machine learning output image. The degree of overlap may be expressed as a numerical value, a ratio, an image, or any other mechanism for assessing degree of similarity or difference. The machine learning model may be further optimized by making adjustments to account for any discrepancies between expected results for the validation data and the output results for the validation data. The accuracy assessment and machine learning optimization may be repeated until the machine learning model outputs results with sufficient degree of accuracy.

In block 1250, the optimized machine learning model may provide output for a task along with a confidence value corresponding to the output. For example, for a task of detecting lumen contours, the confidence value may indicate how likely it is that a portion of the image includes a contour or not.

While the method 1200 is described above in connection with one task, in other examples the confidence value can be obtained based on multiple tasks by integrating the information from each task. The confidence value in either example may be output along with the image frame being assessed. For example, the confidence value may be output as a numerical value on a display. In other examples, the confidence value may be output as a visual, audio, haptic, or other indicator. For example, the indicator may be a color, shading, icon, text, etc. In some examples, the visual indicator may specify a particular portion of the image to which the confidence value corresponds, and a single image may have multiple confidence values corresponding to different portions of the image. For further examples, the indicator may be provided only when the confidence value is above or below a particular threshold. For example, where the confidence value is below a threshold, indicating a low quality image, an indicator may signal to the physician that the image is not sufficiently clear. Where the confidence is above a threshold, the indicator may signal that the image is acceptable. Such thresholds may be determined automatically through the machine learning optimization described above. The image quality indicator not only captures the clarity of image itself, but also brings reliable image characterization results across an entire analysis pipeline, such as for evaluation of medical conditions using a diagnostic medical imaging system.

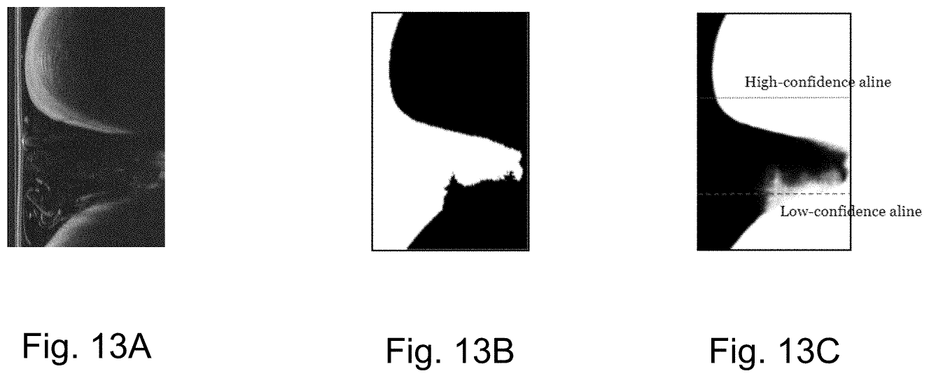

FIGS. 13A-C illustrate an image processed using the machine learning model described above in connection with FIG. 12. In each of FIGS. 13A-C, a horizontal axis indicates a pixel of an A-line, and a vertical axis represents an A-line of an image frame. An A-line may be, for example, a scan line. Where an imaging probe rotates as it passes through the vessel, each rotation may include a plurality of A-lines, such as hundreds of A-lines.

FIG. 13A is an intravascular image, such as an OCT image. FIG. 13B is an output of the machine learning model. For example, for a machine learning model for a lumen detection tasks, the model output may be a binary mask. The white pixels in the binary mask represent the detected lumen, while the back pixels indicate the background. FIG. 13C is a confidence map for the lumen detection. Each pixel is represented by a floating number between 0 and 1, where 0 indicates no confidence and 1 indicates full confidence. The visualization of FIG. 13C reverses the value by (1-confidence value), such that it represents the uncertainty. As shown in FIG. 13C, part of the lumen is out of the field of view, resulting in a low-confidence A-line.

To assess the quality of the image frame, the information embedded in the confidence map may be converted into a binary decision, as a high- or low-quality frame. Given the confidence maps of all the OCT frames, for each frame i, the confidence values of the pixels on each A-line are converted to one single confidence value that represents the quality of entire A-line.

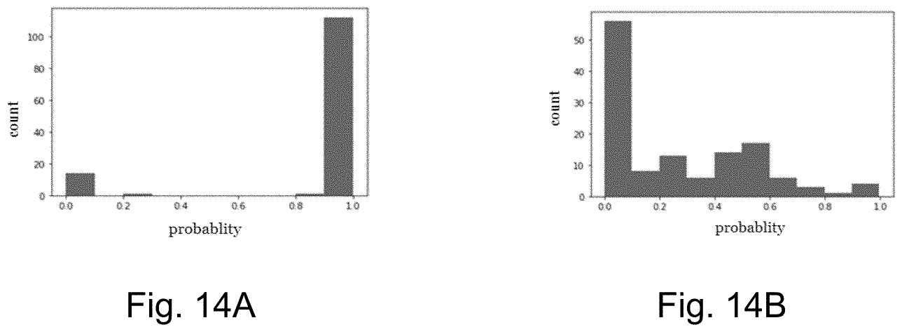

FIGS. 14A-B provide histograms illustrating a difference between high confidence and low confidence quality A-lines. If the lumen detection task identifies a clear segmentation between lumen and not lumen for one A-line, the computational model used in the task will confidently classify pixels on the A-line into either lumen or background. Therefore, the histogram will show that the confidence mostly falls into 0 and 1. However, if the image quality along an A-line is low, the model will be less confident on determining a pixel as lumen or background. The corresponding histogram then clearly visualizes it, where several probability values between 0 and 1 will be presented. Such difference of histograms can be calculated by using entropy defined in the following equation:

E_{i,j}=−Σₐ₌₁ⁿ(p_{a,i,j} log(p_{a,i,j}))

E_{i,j} represents the entropy of the i-th A-line quality at frame j, a is the index of pixel on the i-th A-line, n is the number of pixels on i-th A-line, and p is the probability of the pixel confidence value at location (i, a).

FIG. 14A illustrates an example of entropy on high confidence A-lines. In this example, entropy according to the equation above is 0.48. FIG. 14B illustrates an example of entropy on low confidence A-lines, where entropy is 22.64.

The j-th frame quality may be determined by the following equation:

$${F}_{j} = \{ \begin{matrix}good , if \frac{count ( {E}_{i , j} > {T}_{1} )}{Num\_of \_A - lines} < {T}_{2} \\ bad , otherwise \end{matrix} ,$$

where count is a function calculating the number of A-lines with an entropy value larger than a first threshold T₁. T₂ is second threshold indicating the percentage of A-lines. The first threshold T₁ may be set during manufacture as a result of experimentation. T₁ may be a value between 0 and 1 after normalization of the entropy values. By way of example only, T1 may be 2%, 5%, 10%, 20%, 30%, 50%, or any other value. According to some examples, the value of T1 may be adjusted based on user preference. In this equation, there are “good” and “bad” categories defined for image quality. For example, an image frame may be defined as “good” if the equation results in a value above T2, suggested that a percentage of A-lines above the second threshold have an entropy value above the first threshold, and the image frame may be defined as “bad” if the equation results in a value below T2. In other examples, such confidence analysis may be extended to further identify finer types of categories. For example, “bad” can further include subcategories of occurrence of dissection, sidebranch, thrombus, tangential imaging artifact in OCT, etc.

The value of T2 may be determined, for example, based on receiver operating characteristic (ROC) analysis. For example, the value of T2 may depend on factors or settings that may be defined by a user, such as sensitivity, specificity, positive predictive value, etc. By way of example, if a user prefers to catch every low quality image, sensitivity may be set close to 100% and T2 can be set relatively low, such as between 0-10%. This may result in a higher number of false positives, where image frames are categorized as “bad” when only a few pixels are unclear. In other examples, T2 can be set higher, such as to categorize fewer image frames as “bad.” By way of example only, T2 can be set to approximately 70%, 50%, 30%, 20% or any other value.

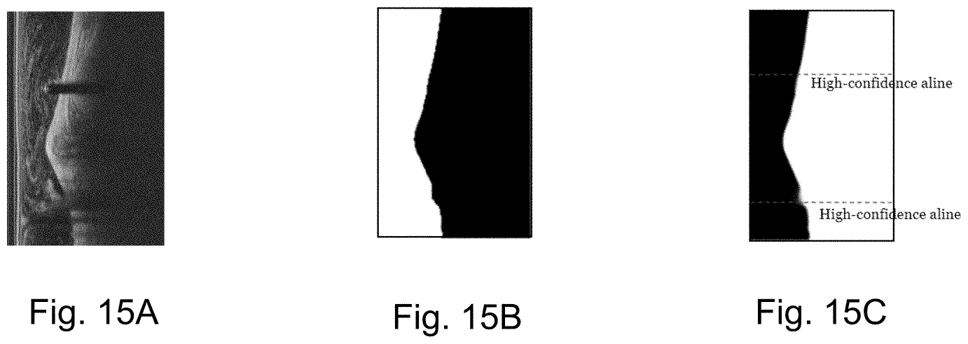

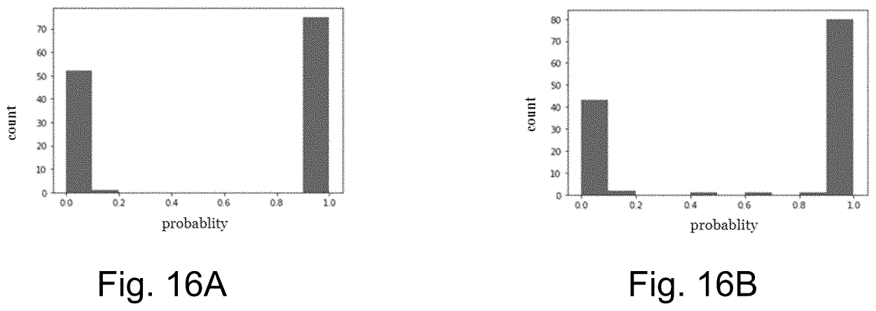

FIGS. 15A-C illustrate another example of image quality detection using a machine learning model. In this example, the obtained image frame illustrated in FIG. 15A is an image with blood artifacts. The segmentation task is accomplished properly even though the blood is spread all over the lumen. Therefore, the mask in FIG. 15B depicts a clear demarcation between the white pixels representing the lumen and the black pixels representing the background. Further, the output of FIG. 15C illustrates high confidence for the detected contours. The model used in this task is robust to the blood artifact, and therefore, the histograms of A-lines in FIGS. 16A-B show that the confidence values mostly fall in the buckets of 0 and 1. The entropy values are low as 0.44 and 2.08. As a result, the frame of FIG. 15A is classified as good quality.

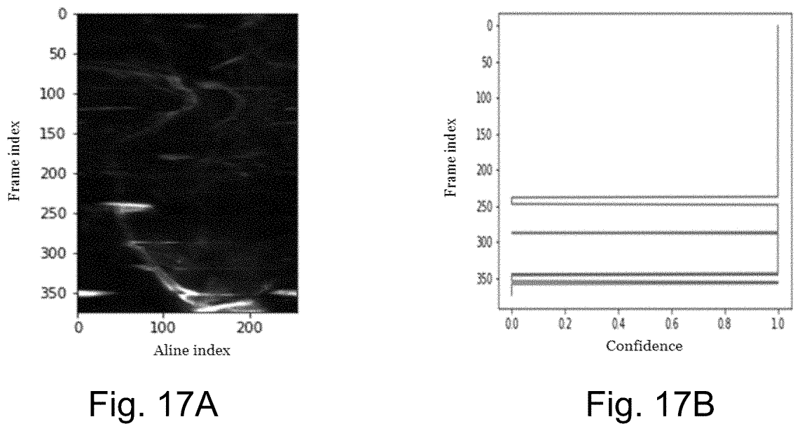

FIGS. 17A-B illustrate an aggregated output of the confidence assessment. FIG. 17A shows the quality of all the A-lines of all the frames in a pullback, where the intensity of a pixel indicates the A-line quality. Using the frame quality, such as determined using the equation above, the OCT image quality can be determined as shown in FIG. 17B, where 0 indicates low quality, and 1 indicates high quality. Certain post-processing can be applied to this result to ensure that the longest clear image length with minimal uncertainty is provided to users.

While the equation above relates to an entropy metric, other metrics may be used. By way of example, such other metrics may include randomness or variation of a data series. The confidence or uncertainty metrics may be calculated from different types of statistics, such as standard deviation, variance, or various forms of entropies, such as Shannon's or computational entropy. The threshold values mentioned above can be determined by either receiver operating characteristic (ROC) analysis, or empirical determination.

According to some examples, image quality indicators matching with the task-based quality metrics may be output. The quality indicators may be, for example, visual, audio, haptic, and/or other types of indicators. For example, the system may play a distinctive audio tone when a captured image meets a threshold quality. As another example, the system may place a visual indicator on a display outputting images obtained during an imaging procedure. In this regard, a physician performing the procedure will immediately know whether sufficient images are obtained, thereby reducing a potential need for a subsequent procedure to obtain clearer images. The reduced need for subsequent procedures results in increased patient safety.

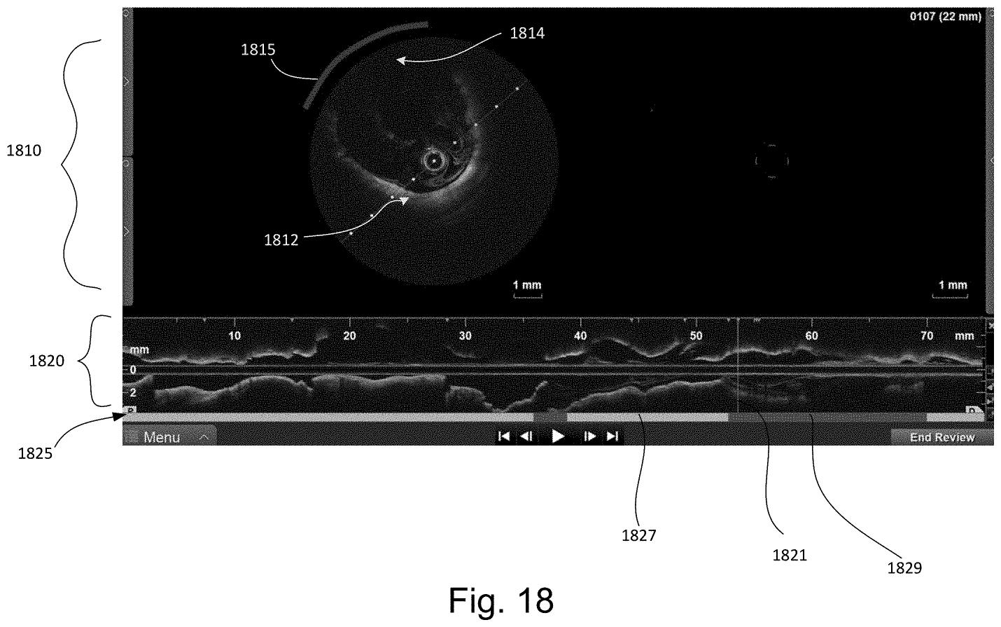

FIG. 18 is a screenshot of an example user interface for an imaging system, the user interface providing visual indications of quality of image frames. The imaging system may be, for example, an intravascular imaging system, such as OCT, ultrasound, NIRS, micro-OCT, etc. In other examples, the real-time quality assessment and indications may be provided for other types of medical or non-medical imaging.

The example of FIG. 18 includes a frame view 1810 and a segment view 1820. The frame view 1810 may be a single image of a plurality of images in the segment view 1820. For example, frame indicator 1821 in the segment view 1820 may identify which frame, relative to other frames in the segment, corresponds to the frame presently depicted in the frame view 1810. In the example of an intravascular imaging procedure, the frame view 1810 may depict a cross-sectional view of the vessel being imaged, while the segment view 1820 depicts a longitudinal view of a segment or portion of the vessel being imaged.

The example of FIG. 18 is for an OCT pullback, where the task is to detect lumen contours. The task may be identified by the physician prior to beginning the pullback, such as by selecting an input option through the user interface. The quality indicators may be specific to the task selected. For example, for a task of detecting lumen contours, the indicators may identify where images or portions of images depicting lumen contours are clear or unclear. For a task of detecting calcium, the indicators may identify where in images calcium is shown relative to a threshold degree of certainty. According to some examples, multiple tasks can be selected, such that the user interface depicts quality indicators relative to the multiple tasks. For example, a first indicator may be provided relative to lumen contours while a second indicator is provided relative to calcium. The first indicator and second indicator may be a same or different types, such as color, gradient, text, annotations, alphanumeric values, etc.

As seen in the frame view 1810, lumen contours are clearly imaged in a first portion 1812 of the image at a lower right-hand side of the image. The lumen contours are less clearly imaged in a second portion 1814 of the image at an upper left-hand side of the image. While the first portion 1812 clearly shows a boundary between lumen walls and the lumen, the second portion 1814 less clearly illustrates the boundary. In this example, a frame view indicator 1815 corresponds to the second portion 1814 in which the lumen contours are not clearly depicted. The frame view indicator 1815 is shown as a colored arc that extends partially around a circumference of the lumen cross-section. An angular distance covered by the arc corresponds to an angular distance of the second portion 1814 in which the lumen contour is not clearly imaged. For example, the frame may be evaluated on a pixel-by-pixel basis, such that image quality can be assessed for each pixel, and quality indicators can correspond to particular pixels. Accordingly, the frame indicator 1815 can identify the specific portions of the image for which the image quality is below a particular threshold.

While the frame quality indicator 1815 is shown as a colored arc, it should be understood that any of a variety of other types of indicators may be used. By way of example only, such other types of indicators may include but not be limited to an overlay, annotation, shading, text, etc. According to some examples, the indicator may depict a degree of quality for different portions of the image. For example, the arc in FIG. 18 can be a gradient of color, shade, degree of transparency, or the like, where one end of a spectrum corresponds to a lower quality and another end of the spectrum corresponds to a higher quality.

The segment view 1820 may also include an indicator of quality. As shown, segment quality indicator 1825 may indicate a quality of each image frame along the imaged vessel segment. In the example of FIG. 18, the segment quality indicator 1825 is a colored bar that extends along a length of the segment view. The colored bar includes a first color indicating where frame quality is above a threshold and a second color indicating where frame quality is below a threshold. For example, the threshold may correspond to a portion or percentage of each frame for which images according to the task were captured with sufficient clarity. Such threshold may correspond, for example, to the threshold T₂ described in connection with the frame quality equation above. In this example, first portion 1827 of the segment quality indicator 1825 is a first color, corresponding to frames in the segment having a sufficient quality, above a threshold. Second portion 1829 of the segment quality indicator 1825 is a second color, corresponding to frames in the segment have lower quality, below the threshold, such as the frame illustrated in frame view 1810. While the segment quality indicator 1825 in this example distinguishes the quality of each frame along the segment using color, in other examples the segment quality indicator 1825 may use other indicia, such as shading, gradient, annotations, etc. Moreover, while the segment quality indicator 1825 is shown as a bar, it should be understood that any other shape, size, or form of indicia may be used.

While some examples above are described in connection with OCT imagery, the techniques of automatic real-time quality detection, using direct deep learning or lumen confidence, as described above may be applied in any of a variety of medical imaging modalities, including but not limited to IVUS, NIRS, micro-OCT, etc. For example, a machine learning model for lumen detection may be trained using IVUS images having annotated lumens. The confidence signal from that model may be used to gauge image quality. As another example, the IVUS frames may be annotated as high or low quality, and the direct deep learning approach of detecting image quality may be applied in real-time image acquisition during an IVUS procedure. As yet another example, when using high-definition intravascular ultrasound (HD-IVUS), a saline flush may be used to clear blood to provide improved IVUS image quality. In such cases, the quality detection techniques may be applied to distinguish between flushed and non-flushed regions of the vessel. In further examples, the quality detection techniques may be based on IVUS parameters such as grayscale or axial/lateral resolution. For example, the machine learning model may be trained to detect whether images are obtained with a threshold resolution. It should be understood that any of a variety of further applications of the techniques described herein are also possible.

Aspects of the disclosed technology can include the following combination of features:

Feature 1. A method of classifying a diagnostic medical image, the method comprising:

receiving the diagnostic medical image;

analyzing, in real time or near real time, with a trained machine learning model, the diagnostic medical image, wherein the trained machine learning model is trained on a set of annotated diagnostic medical images;

identifying, based on the analyzing, an image quality for the diagnostic medical image; and

outputting for display on a user interface, in real time or near real time, an indication of the identified image quality.

Feature 2. The method of feature 1 wherein the diagnostic medical image is a single image of a series of diagnostic medical images.

Feature 3. The method of features 2 wherein the series of diagnostic medical images is obtained through an optical coherence tomography pullback.

Feature 4. The method of feature 1 further comprising classifying the diagnostic medical image as a first classification or a second classification.

Feature 5. The method of features 1-4 further comprising providing an alert or notification when the diagnostic medical image is classified in the second classification.

Feature 6. The method of feature 1 wherein the set of annotated diagnostic medical images comprises annotations including clear, blood, or guide catheter.

Feature 7. The method of feature 1 wherein the diagnostic medical image is an optical coherence tomography image.

Feature 8. The method of feature 1 further comprising classifying the diagnostic medical image as a clear medical image or a blood medical image.

Feature 9. The method of feature 1 further comprising computing a probability indicative of whether the diagnostic medical image is acceptable or not acceptable.

Feature 10. The method of feature 9 further comprising using a threshold method to convert the computed probability to a classification of the diagnostic medical image.

Feature 11. The method of feature 9 further comprising using graph cuts to convert the computed probability to a classification of the diagnostic medical image.

Feature 12. The method of features 1-9 further comprising using morphological classification to convert the computed probability to a classification of the diagnostic medical image.

Feature 13. The method of features 1-9 wherein acceptable means that the diagnostic medical image is above a predefined threshold quality which allows for evaluation of characteristics of human tissue above a threshold level of accuracy or confidence.

Feature 14. A system comprising a processing device coupled to a memory storing instructions, the instructions causing the processing device to:

receive the diagnostic medical image;

analyze, in real time or near real time, with a trained machine learning model, the diagnostic medical image, wherein the trained machine learning model is trained on a set of annotated diagnostic medical images;

identify, based on the analyzing, an image quality for the diagnostic medical image; and

output for display on a user interface, in real time or near real time, an indication of the identified image quality.

Feature 15. The system of feature 14 wherein the diagnostic medical image is an optical coherence tomography (OCT) image.

Feature 16. The system of feature 15 wherein the instructions are configured to display a plurality of OCT images along with an indicator associated with a classification of each image of the plurality of OCT images.

Feature 17. The system of features 14-16 wherein the series of diagnostic medical images is obtained through an optical coherence tomography pullback.

Feature 18. A non-transitory computer readable medium containing program instructions, the instructions when executed perform the steps of:

receiving the diagnostic medical image;

analyzing, in real time or near real time, with a trained machine learning model, the diagnostic medical image, wherein the trained machine learning model is trained on a set of annotated diagnostic medical images;

identifying, based on the analyzing, an image quality for the diagnostic medical image; and

outputting for display on a user interface, in real time or near real time, an indication of the identified image quality.

Feature 19. The non-transient computer readable medium of feature 18 wherein the diagnostic medical image is a single image of a series of diagnostic medical images.

Feature 20. The non-transient computer readable medium of feature 19 wherein the series of diagnostic medical images is obtained through an optical coherence tomography pullback.

Feature 21. The non-transient computer readable medium of features 18-20 further comprising classifying the diagnostic medical image as a first classification or a second classification.

Feature 22. The non-transient computer readable medium of features 18-21 further comprising providing an alert or notification when the diagnostic medical image is classified as the second classification.

Feature 23. The non-transient computer readable medium of features 18-22 wherein the set of annotated diagnostic medical images comprises annotations including clear, blood, or guide catheter.

Feature 24. The non-transient computer readable medium of features 18-22 wherein the diagnostic medical image is an optical coherence tomography image.

Feature 25. The non-transient computer readable medium of features 18-24 further comprising classifying the diagnostic medical image as a clear medical image or a blood medical image.

Feature 26. The non-transient computer readable medium of feature 18 further comprising computing a probability indicative of whether the diagnostic medical image is acceptable or not acceptable.

Feature 27. The non-transient computer readable medium of features 18-26 further comprising using a threshold non-transient computer readable medium to convert the computed probability to a classification of the diagnostic medical image.

Feature 28. The non-transient computer readable medium of feature 27 further comprising storing an unclassifiable image to retrain the trained machine learning model.

Feature 29. The non-transient computer readable medium of feature 18 further comprising outputting a clear image length or clear image length indicator.

Feature 30. The system of feature 14 wherein the instructions are configured to display a clear image length or clear image length indicator.

Feature 31. The method of feature 1 further comprising displaying or outputting a clear image length or clear image length indicator.

The aspects, embodiments, features, and examples of the disclosure are to be considered illustrative in all respects and are not intended to limit the disclosure, the scope of which is defined only by the claims. Other embodiments, modifications, and usages will be apparent to those skilled in the art without departing from the spirit and scope of the claimed disclosure.

The use of headings and sections in the application is not meant to limit the disclosure; each section can apply to any aspect, embodiment, or feature of the disclosure

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” or “substantially” is before a quantitative value, the present teachings also include the specific quantitative value itself, unless specifically stated otherwise. The terms “about” and “substantially” as used herein, refer to variations in a numerical quantity that can occur, for example, through measuring or handling procedures in the real world; through inadvertent error in these procedures; through differences/faults in the manufacture of materials, such as composite tape, through imperfections; as well as variations that would be recognized by one in the skill in the art as being equivalent so long as such variations do not encompass known values practiced by the prior art. Typically, the terms “about” and “substantially” means greater or lesser than the value or range of values stated by 1/10 of the stated value, e.g., ±10%.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

It is to be understood that the figures and descriptions of the disclosure have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

1. A method of classifying a diagnostic medical image, the method comprising:
receiving the diagnostic medical image;
analyzing, in real time or near real time, with a trained machine learning model, the diagnostic medical image, wherein the trained machine learning model is trained on a set of annotated diagnostic medical images;
identifying, based on the analyzing, an image quality for the diagnostic medical image; and
outputting for display on a user interface, in real time or near real time, an indication of the identified image quality.

2. The method of claim 1 wherein the diagnostic medical image is a single image of a series of diagnostic medical images.

3. The method of claim 2 wherein the series of diagnostic medical images is obtained through an optical coherence tomography pullback.

4. The method of claim 1 further comprising classifying the diagnostic medical image as a first classification or a second classification.

5. The method of claim 4 further comprising providing an alert or notification when the diagnostic medical image is classified in the second classification.

6. The method of claim 1 wherein the set of annotated diagnostic medical images comprises annotations including clear, blood, or guide catheter.

7. The method of claim 1 wherein the diagnostic medical image is an optical coherence tomography image.

8. The method of claim 1 further comprising classifying the diagnostic medical image as a clear medical image or a blood medical image.

9. The method of claim 1 further comprising computing a probability indicative of whether the diagnostic medical image is acceptable or not acceptable.

10. The method of claim 9 further comprising using a threshold method to convert the computed probability to a classification of the diagnostic medical image.

11. The method of claim 9 further comprising using graph cuts to convert the computed probability to a classification of the diagnostic medical image.

12. The method of claim 9 further comprising using morphological classification to convert the computed probability to a classification of the diagnostic medical image.

13. The method of claim 9 wherein acceptable means that the diagnostic medical image is above a predefined threshold quality which allows for evaluation of characteristics of human tissue above a threshold level of accuracy or confidence.

14. The method of claim 13, wherein a value for the predefined threshold quality is determined by optimizing a machine learning model.

15. A system comprising a processing device coupled to a memory storing instructions, the instructions causing the processing device to:
receive the diagnostic medical image;
analyze, in real time or near real time, with a trained machine learning model, the diagnostic medical image, wherein the trained machine learning model is trained on a set of annotated diagnostic medical images;
identify, based on the analyzing, an image quality for the diagnostic medical image; and
output for display on a user interface, in real time or near real time, an indication of the identified image quality.

16. The system of claim 15 wherein the diagnostic medical image is an optical coherence tomography (OCT) image.

17. The system of claim 16 wherein the instructions are configured to display a plurality of OCT images along with an indicator associated with a classification of each image of the plurality of OCT images.

18. The system of claim 15 wherein the series of diagnostic medical images is obtained through an optical coherence tomography pullback.

19. A non-transitory computer readable medium containing program instructions, the instructions when executed perform the steps of:
receiving the diagnostic medical image;
analyzing, in real time or near real time, with a trained machine learning model, the diagnostic medical image, wherein the trained machine learning model is trained on a set of annotated diagnostic medical images;
identifying, based on the analyzing, an image quality for the diagnostic medical image; and
outputting for display on a user interface, in real time or near real time, an indication of the identified image quality.

20. The non-transitory computer readable medium of claim 19 wherein the diagnostic medical image is a single image of a series of diagnostic medical images.
